(19) United States
(12) Plant Patent Application Publication
(10) Pub. No.: US 2015/0208568 P1
(43) Pub. Date: Jul. 23, 2015

(54) Senecio cruentus plant named 'Sunsenebubakai'

(50) ;

(71) Applicant: SUNTORY FLOWERS LIMITED, TOKYO (JP)

(72) Inventor: Tomoya Misato, Omihachiman-shi (JP)

(21) Appl. No.: 14/121,584

(22) Filed: Sep. 19, 2014

Related U.S. Application Data
(60) Provisional application No. 61/965,150, filed on Jan. 23, 2014.

(51) Int. Cl. A01H 5/00

(52) U.S. Cl. PLT/480

(57) ABSTRACT

Disclosed herein is a new and distinct variety of Senecio plant having blue and white-bicolored ray floerts, and upright and medium sized appearance.

### CROSS REFERENCE TO RELATED APPLICATIONS

This application is related to and claims priority under 35 U.S.C. 119 to U.S. provisional plant patent application 61/965,150, filed Jan. 23, 2014, entitled ‘Sunsenebubakai.’

### BOTANICAL DESIGNATION

Senecio cruentus (Masson ex L′Hér.) DC.

### CULTIVAR DENOMINATION

‘Sunsenebubakai’

### BACKGROUND OF THE INVENTION

The present invention relates to a new and distinct variety of Senecio plant, which is hereinafter referred to as ‘Sunsenebubakai.’

A Senecio cruentus (Masson ex L′Hér.) DC. is a very popular plant that is used in flower bedding and potting in the winter and spring seasons.

The purpose of this invention is to obtain a new Senecio cultivar with abundant and large sized petals having blue and white-bicolored ray floerts, and an upright and medium sized appearance.

The new Senecio plant originated from cross-pollination of the female parent ‘09-83’ and the male parent ‘09-09’. The female parent ‘09-83’ (unpatented) used in the crossing of ‘Sunsenebubakai’ is a strain of the applicant's breeding lines (i.e., proprietary Senecio selection), having white petals, and the male parent ‘09-09’ (unpatented) used in the crossing of ‘Sunsenebubakai’ is a strain of the applicant's breeding lines (i.e., proprietary Senecio selection), having light blue and pink-bicolored petals. The cross-pollination was conducted in March, 2010, at Higashiomi, Shiga, Japan.

In February 2011, the seedlings obtained by the crossing were planted in a field, and some seedlings were selected in view of growth habit, flower size and color thereof. Shortly thereafter stem tip culturing was carried out, and then the propagation was started.

In November 2013, the cultivation of the seedlings was repeated. The botanical characteristics of that plant were then examined, using similar variety ‘Sunsenebuba’ for comparison. As a result, it was concluded that this Senecio plant is distinguishable from any other variety, whose existence is known, and has uniform and stable characteristics.

The new variety of Senecio plant was then named ‘Sunsenebubakai’.

### SUMMARY OF THE INVENTION

This new variety is unlike any Senecio commercially available as evidenced by the following unique combinations of characteristics.

1. Abundant and large sized petals having blue and white-bicolored ray floerts.

2. Upright and medium sized appearance.

The new variety ‘Sunsenebubakai’ differs from the similar variety ‘Sunsenebuba’, which was applied for Japanese plant variety protection (the application number: 12095; the application date: 199/11/10; the registration number 10653; the registration date: 2002Sep. 31), in the following points.

1. The ray floret color of ‘Sunsenebubakai’ is bicolor, blue (95B) and white (NN155C). That of ‘Sunsenebuba’ is bicolor, violet-blue (93B) and white (NN155C).

2. The flower diameter of ‘Sunsenebubakai’ is about 81 mm. That of ‘Sunsenebuba’ is about 68 mm.

3. Ray floret length of ‘Sunsenebubakai’ is about 31 mm. That of ‘Sunsenebuba’ is about 27 mm.

4. The disc diameter of ‘Sunsenebubakai’ is about 16 mm. That of ‘Sunsenebuba’ is about 12 mm.

This new variety of Senecio plant ‘Sunsenebubakai’ was asexially reproduced by the use of cuttings at Higashiomi, Shiga, Japan, and homogeneity and stability thereof were confirmed. The instant plant retains its distinctive characteristics and produces true to type in successive generations.

### BRIEF DESCRIPTION OF THE PHOTOGRAPHS

The depicted plants had been reproduced by the use of cuttings and were photographed during November 2013 while growing outdoors in wall pots which were about 15 cm in size at an age of approximately 6 months.

FIG. 1 is a photograph of a typical plant of the new variety of Senecio plant ‘Sunsenebubakai’ while growing in a pot.

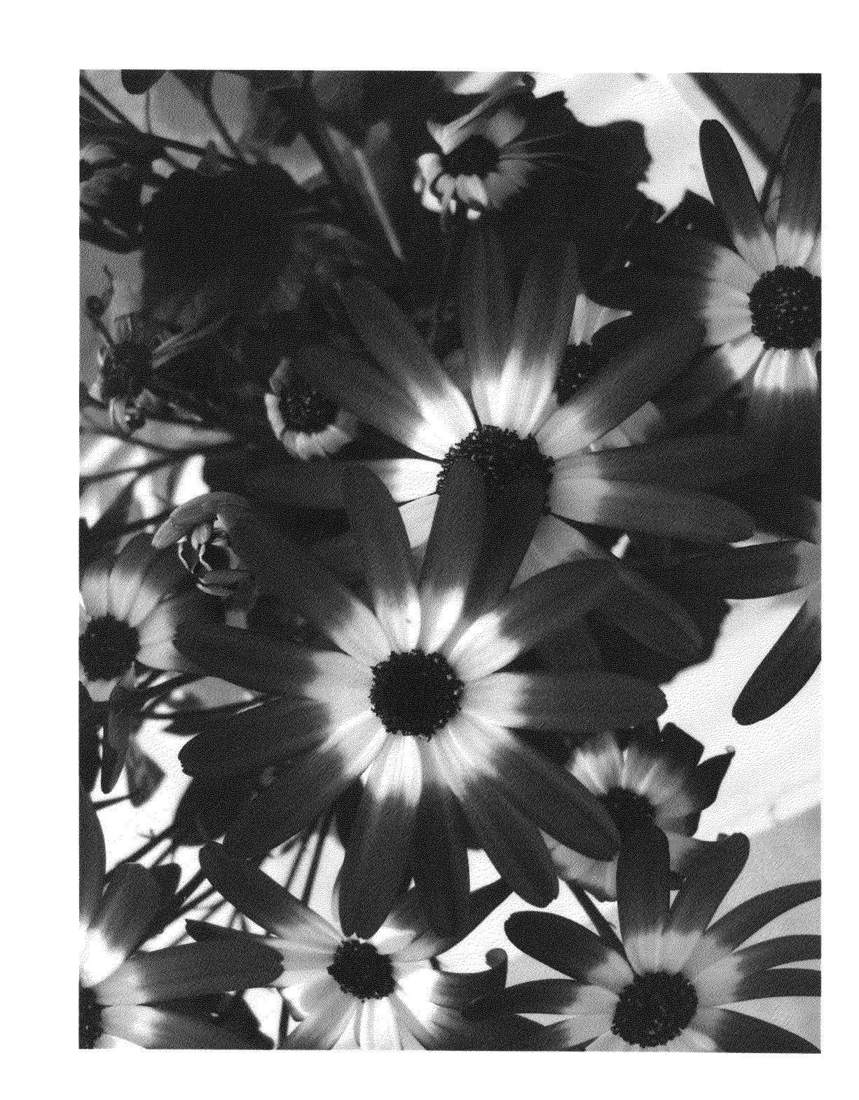

FIG. 2 is a photograph of a close view of flowers of the new variety of Senecio plant ‘Sunsenebubakai’.

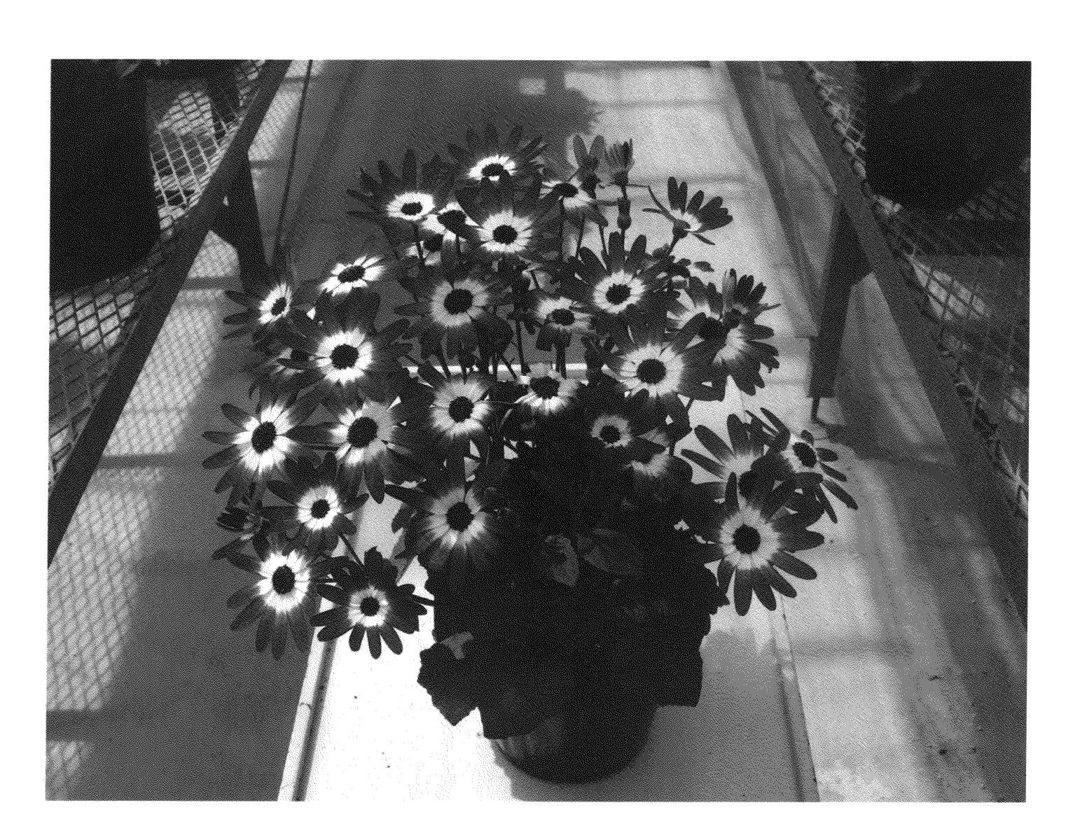

### DETAILED BOTANICAL DESCRIPTION

In November 2013, the cultivation of the seedlings was repeated at Higashiomi, Shiga, Japan. The average day temperature was about 10° C., and the average night temperature was about 5° C. The plants were grown under natural sunlight. The number of days to flowering (response time) was about 22 weeks. The quality was maintained for about 150 days. The plants had temperature resistance to about 0° C. (the lowest temperature) and about 30° C. (the highest temperature). Further, the plants had the same tolerance to pests and pathogens as a typical Senecio plant.

For the parentage information: The female parent ‘09-83’ (unpatented) used in the crossing of ‘Sunsenebubakai’ is a strain of the applicant's breeding lines (i.e., proprietary selection of Senecio cruentus), having white petals, and the male parent ‘09-09’ (unpatented) used in the crossing of ‘Sunsenebubakai’ is a strain of the applicant's breeding lines (i.e., proprietary selection of Senecio cruentus), having light blue and pink-bicolored petals.

For the propagation information: the number of days in which roots started to form during the summer was about 7 days; approximate soil and/or air temperature during the summer was around 18-20° C.; number of days in which roots started to form during the winter was about 7 days; approximate soil and/or air temperature during the winter was around 18-20° C.; number of days to produce a rooted young plant during the summer was about 28 days; number of days to produce a rooted young plant during the winter was about 28 days; root density was fine; root branching was free; root color was white; and root texture was fibrous.

The botanical characteristics of the new and distinct variety of Senecio plant named ‘Sunsenebubakai’ at an age of approximately 6 months are shown in the following Table. In the following description, the color-coding is in accordance with the Horticultural Colour Chart of The Royal Horticultural Society, London, England (R.H.S. Colour Chart 5th edition 2007).

PLANT VARIETY DESCRIPTION

| | APPLICATION VARIETY |
|---|---|
| CHARACTERISTIC | Sunsenebubakai |
| ANNUAL, BIENNIAL or | Grown as annual |
| PERENNIAL? | |
| TYPE OF PLANT: i.e., TREE, | Potted plant |
| SHRUB, SUBSHRUB, VINE, CUT | |
| FLOWER, POTTED PLANT | |
| APPROPRIATE CONTAINERS +/or | Ideal for pots |
| CROPPING SYSTEM | |
| GROWTH HABIT | Upright |
| PLANT HEIGHT | About 47.0 cm |
| PLANT DIAMETER OR AREA OF | About 57.0 cm |
| SPREAD | |
| PLANT VIGOR | Vigorous |
| BRANCHING HABIT | Freely branching |
| PINCHING REQUIRED? | Not required, 1~2 |
| | pinching stimulate |
| | brunching |
| NUMBER OF LATERAL BRANCHES | About 8 |
| LATERAL BRANCH LENGTH | About 29.5 cm |
| LATERAL BRANCH DIAMETER | About 7.1 mm |
| INTERNODE LENGTH | About 15.7 mm |
| STEM ASPECT | Upright |
| STEM COLOR (and bark color, | Near 143A RHS |
| if applicable) | |
| STEM COLOR (anthocyanin | Near - RHS |
| coloration) | |
| STEM PUBESCENCE? | Pubescent |
| OTHER PLANT/STEM | — |
| CHARACTERISTICS | |
| LEAF ARRANGEMENT | Alternate |
| COMPOUND OR SIMPLE? | Simple |
| LEAF (LEAFLET) SHAPE | Cordate |
| LEAF (LEAFLET) TIP | Acute |
| LEAF (LEAFLET) BASE | Cordate |
| LEAF LENGTH | About 64.3 mm |
| LEAF WIDTH | About 72.9 mm |
| LEAF THICKNESS | About 1.1 mm |
| LEAF (LEAFLET) TEXTURE | Smooth |
| LEAF PUBESCENCE? | (Upper side) Dense |
| LEAF PUBESCENCE COLOR | Near - RHS |
| LEAF (LEAFLET) MARGIN | Palmately lobed, |
| | dentate |
| DEGREE OF SERRATION | Shallow |
| UNDULATION | Undulated, weak |
| VENATION PATTERN | Reticulate venation |
| LEAF COLOR, IMMATURE, UPPER | Near 137C RHS |
| SIDE | |
| LEAF COLOR, IMMATURE, LOWER | Near 93C RHS |
| SIDE | |
| LEAF COLOR, MATURE, UPPER | Near 137B RHS |
| SIDE | |
| LEAF COLOR, MATURE, LOWER | Near 191A RHS |
| SIDE | |
| VENATION COLOR, UPPER SIDE | Near 144A RHS |
| VENATION COLOR, LOWER SIDE | Near 144A RHS |
| PETIOLE LENGTH | About 50.0 mm |
| PETIOLE DIAMETER | About 2.4 mm |
| PETIOLE TEXTURE | Tomentose |
| PETIOLE COLOR | Near 144A RHS |
| WING | Absent |
| WING LENGTH | About - mm |
| WING DIAMETER | About - mm |
| STIPULES, TENDRILS, THORNS, | |
| SPINES OR PRICKLES? IF SO, | Absent |
| GIVE COLOR AND SIZE | |
| OTHER FOLIAGE CHARACTERISTICS | — |
| FLOWER ARRANGEMENT | Daisy-type |
| | inflorescence borne in |
| | upper leaf |
| INFLORESCENCE TYPE OR FORM | Mounding |
| (if applicable) | |
| INFLORESCENCE DIAMETER | About 22.0 cm |
| INFLORESCENCE HEIGHT | About 26.0 cm |
| FLOWER TYPE or FORM | Single |
| Continuous flowering - | |
| FLOWERING HABIT | Buds grow one after |
| another from axils. | |
| QUANTITY OF FLOWERS and BUDS | About 144 |
| PER PLANT | |
| QUANTITY OF FLOWERS PER | About 6 |
| INFLORESCENCE | |
| NATURAL FLOWERING SEASON | Substantially |
| | continuous blooming |
| | from winter to late |
| | spring |
| TIME TO FLOWER OR RESPONSE | About 24 weeks |
| TIME | |
| FRAGRANCE | Absent |
| FLOWER BUD LENGTH | About 7.3 mm |
| FLOWER BUD DIAMETER | About 6.3 mm |
| FLOWER BUD SHAPE | Globose |
| FLOWER BUD COLOR | Near 144A RHS |
| FLOWER ASPECT; i.e., UPRIGHT, | Upright |
| OUTWARD, DROOPING, etc. | |
| FLOWER SHAPE | Daisy like shape |
| FLOWER DIAMETER | About 81.0 mm |
| FLOWER DEPTH (HEIGHT) | About 12.0 mm |
| FLOWER LONGEVITY ON PLANT | About 2 weeks at around 15° C. |
| PERSISTENT OR SELF-CLEANING? | Persistent |
| *RAY FLORETS | |
| RAY FLORET TEXTURE, UPPER | Velvety |
| SURFACE. | |
| RAY FLORET TEXTURE, LOWER | Smooth |
| SURFACE | |
| RAY FLORET ARRANGEMENT | Daisy type |
| RAY FLORET NUMBER | About 13 |
| RAY FLORET SHAPE | Narrow elliptic |
| RAY FLORET MARGIN | Entire |
| RAY FLORET TIP | Bitten |
| RAY FLORET BASE | — |
| RAY FLORET LENGTH | About 31.0 mm |
| RAY FLORET WIDTH | About 7.9 mm |
| RAY FLORET COLOR , WHEN | Near Main N89A Center RHS |
| OPENING, UPPER SIDE | NN155C |
| RAY FLORET COLOR , WHEN | Near - RHS |
| OPENING, LOWER SIDE | |
| RAY FLORET COLOR, FULLY | Near Main 95B Center |
| OPENED, UPPER SIDE | NN155C RHS |
| RAY FLORET COLOR, FULLY | Near - RHS |
| OPENED, LOWER SIDE | |
| RAY FLORET COLOR, FADING TO | Near - RHS |
| *DISC FLORET | |
| DISC DIAMETER | About 16.0 mm |
| DISC COLOR (DISC FLORET | Near N89C RHS |
| IMMATURE, MATURE) | |
| DISC FLORET NUMBER | About 137 |
| DISC FLORET SHAPE | Tubular |
| DISC FLORET TIP | 5 pointed |
| DISC FLORET BASE | Fused |
| DISC FLORET LENGTH | About 7.2 mm |
| DISC FLORET DIAMETER | About 1.2 mm |
| INVOLUCURE SHAPE | Cup shape |
| INVOLUCURE TEXTURE | Smooth |
| INVOLUCURE COLOR | Near 143A RHS |
| INVOLUCURE LENGTH | About 7 mm |
| INVOLUCURE DIAMETER | About 8.4 mm |
| INVOLUCURE ARRANGEMENT | Single whorl, fused |
| NUMBER OF INVOLUCURAL BRACTS | About 15 |
| (PHYLLARIES) | |
| INVOLUCURAL BRACT (PHYLLARY) | Lanceolate |
| SHAPE | |
| INVOLUCURAL BRACT (PHYLLARY) | Entire |
| MARGIN | |
| INVOLUCURAL BRACT (PHYLLARY) | Narrow acute |
| TIP | |
| INVOLUCURAL BRACT (PHYLLARY) | Fused |
| BASE | |
| INVOLUCURAL BRACT (PHYLLARY) | Smooth |
| TEXTURE | |
| INVOLUCURAL BRACT (PHYLLARY) | About 8.5 mm |
| LENGTH | |
| INVOLUCURAL BRACT (PHYLLARY) | About 2.2 mm |
| WIDTH | |
| INVOLUCURAL BRACT (PHYLLARY) | Near 144A RHS |
| COLOR, UPPER SIDE | |
| INVOLUCURAL BRACT (PHYLLARY) | Near 144C RHS |
| COLOR, LOWER SIDE | |
| PEDUNCLE LENGTH | About 32.3 mm |
| PEDUNCLE DIAMETER | About 2.9 mm |
| PEDUNCLE ANGLE | Upright to semi- |
| | upright |
| PEDUNCLE TEXTURE | Smooth |
| PEDUNCLE COLOR | Near 144A RHS |
| STAMEN NUMBER | 5 per a disc floret |
| ANTHER SHAPE | Ellipsoidal |
| ANTHER COLOR | Near N186A RHS |
| AMOUNT OF POLLEN | |
| POLLEN COLOR | Near 7B RHS |
| PISTIL NUMBER | 1 per a ray and disc |
| | floret |
| STIGMA SHAPE | Bi-parted |
| STIGMA COLOR | Near N186A RHS |
| OTHER FLOWER CHARACTERISTICS | — |
| QUANTITY OF SEEDS | Seed production has |
| | not been observed. |
| ROOT STRUCTURES such as | Fibrous root |
| BULBS, CORMS or RHIZOMES? | |
| LOW TEMPERATURE TOLERANCE | 0° C. (However, the |
| | plant would be |
| | seriously damaged by |
| | frost, as other |
| | Senecio Plant, at any |
| | temperature.) |
| HIGH TEMPERATURE TOLERANCE | Normal |
| DISEASE RESISTANCE AND/OR | Normal |
| SUSCEPTIBILITY | |
| PEST RESISTANCE AND/OR | Normal |
| SUSCEPTIBILITY | |

This new variety of Senecio plant having the above botanical characteristics is suitable for flower bedding and potting, particularly in hanging pots or planters.

What is claimed:

1. A new and distinct variety of Senecio plant named ‘Sunsenebubakai’, substantially as herein illustrated and described.
